(19) United States
(12) Patent Application Publication
(10) Pub. No.: US 2010/0168773 A1
(43) Pub. Date: Jul. 1, 2010

(54) GUARDED SURGICAL SCALPEL WITH MEANS FOR MOUNTING A BLADE THEREON (AND SUBSEQUENTLY REMOVING A USED BLADE) AND WITH FURTHER MEANS FOR CLEANING AND STERILIZING THE SCALPEL FOLLOWING A SURGICAL PROCEDURE

(76) Inventors: Robert V. Funderburk, Salisbury, NC (US); John F. Higdon, Matthews, NC (US); Julie C. Howell, Indian Trail, NC (US); Steven P. Lehmbeck, Baltimore, MD (US)

Correspondence Address: Robert M Gamson;Hodes Pessin & Katz, 901 Dulaney Valley Road, Suite 400, Towson, MD, 21204, US

(21) Appl. No.: 12/449,943

(22) PCT Filed: Mar. 4, 2008
(86) PCT No.: PCT/US08/02843

§ 371 Date: Feb. 4, 2010

Related U.S. Application Data
(60) Provisional application No. 60/904,923, filed on Mar. 5, 2007.
(60) Provisional application No. 61/003,928, filed on Nov. 21, 2007.

(51) Int. Cl. A61B 17/3213

(52) U.S. Cl. 606/167

(57) ABSTRACT

A guarded surgical scalpel is provided with manually-manipulatable means carried by the scalpel for, first, securely mounting a surgical blade on the scalpel and, second, subsequently de-mounting the surgical blade off the scalpel, the de-mounting being essentially in a “hands free” environment; that is, it is not necessary to touch the used blade for its removal. Additionally, the scalpel may be quickly disassembled for cleaning and/or sterilization following a surgical procedure in the operating room (“O.R.”) of a hospital, clinic or physician's office.

### CROSS REFERENCE TO RELATED APPLICATIONS

The present invention incorporates the disclosures and teachings of provisional application, Ser. No. 60/904,923 filed Mar. 5, 2007 and Ser. No. 61/003,928 filed Nov. 21, 2007, respectively, and discloses and claims further improvements thereon.

### BACKGROUND OF THE INVENTION

One of the inventors (applicant) herein, Mr. Steven P. Lehmbeck , along with his colleague, Dr. Michael R. Abidin, were the basic inventors and pioneers of a practical guarded surgical scalpel to prevent accidental and inadvertent cuts that may be incurred during a surgical procedure in the operating room of a hospital or clinic. See Abidin, et. Al. U.S. Pat. Nos. 5,250,063; 5,275,606; 5,411,512; and 5,569,281.

Accordingly, the blade was exposed only during its actual use, and the blade was covered while being transferred in the operating room from the nurse or technician to the surgeon and vice-versa.

The blade, however, was still manually mounted on the scalpel and was subsequently demounted in the conventional manner. Surgical scalpel blades, of course, are extremely sharp and can cause a very deep and painful accidental cut.

Realizing that the exposure to a used blade was more serious, especially if the patient had an infectious disease (such as H.I.V.), these same inventors, Mssrs. Abidin and Lehmbeck, were the first to disclose a practical means for stripping a used blade off the scalpel. This stripping means comprised a wedge that would be moved forwardly (and independently of the blade carrier) to lift the rearward edge of the blade—laterally of the blade carrier and in a camming action—after which the guard on the scalpel was moved forwardly to engage the lifted-away rearward edge of the blade and eject the blade away from the scalpel. [See Abidin, et. al. U.S. Pat. Nos. 5,496,340 and 5,662,669.]

Thereafter, one manufacturer in the medical products industry followed this disclosure and came up with a variation of the inventor's original unique concept of a guarded surgical scalpel equipped with a blade stripper.

To prevent an accidental or inadvertent ejection of the blade, Abidin and Lehmbeck expanded the cutting edge of the technology still further and invented a stripper lock as disclosed and claimed in pending application Ser. No. 10/670,478 filed Sep. 24, 2003.

Sometime thereafter, new government standards were adopted by the Occupational Safety and Health Agency (see OSHA standard No. 1910.1030 bloodborne pathogens).

Needless to say, these OSHA standards achieved their intended purpose—they got the various manufacturers' attention—and the medical products industry, particularly, accelerated their product development to meet these new standards.

Accordingly, one of the leading medical products manufacturers subsequently marketed a guarded scalpel together with a blade package, but this development had several inherent problems: First, you must buy both the handle and the blade. Their guarded handle is special and cannot be used with a conventional surgical blade, and their blade package itself is special and cannot be used with any other scalpel handle (guarded or unguarded). Secondly, their special blade package costs around four or five times the cost of a conventional surgical blade.

In an era where hospital administrators are strenuously attempting to standardize, to use up existing inventory of supplies, and to keep the lid on soaring costs, this product development—while perhaps interesting purely from a design standpoint—leaves much to be desired.

Some others in the medical products industry have come up with other schemes which, while perhaps somewhat interesting, have inherent design flaws which may actually precipitate an injury in the O.R.

For example, in one manufacturer's design, the blade carrier is extended beyond the housing (the handle); the blade is placed on the extended portion of the blade carrier; and the blade carrier is retracted within the housing so that the blade is firmly secured in the blade carrier by a pinching or camming action.

This scalpel has these detented positions (1) blade retracted, which is the guarded position; (2) the blade is extended beyond the housing for use during the surgical procedure, and (3) the blade is further extended beyond the housing (beyond the “in-use” position of the blade), which is basically its original position. In this third (further extended) position, the blade may freely fall off the scalpel.

The problem with this design is as follows: In actual use in the O.R., if you push the button a little too vigorously, you can easily override the second detented position and go past step (2) and into step (3) directly, in which case the blade could accidentally wind up in the operative site, that is, within the patient's body. Obviously, this is totally unacceptable.

Accordingly, a need still exists for a guarded surgical scalpel which first, meets OSHA's criteria and standards and, secondly, is safe, practical, inherently low cost, and will accommodate standard conventional surgical blades.

In this regard, the applicants' market research shows that, worldwide, around one billion (1,000,000,000) surgical scalpel blades are sold each year, with around 40% or so sold in the United States.

### SUMMARY OF THE INVENTION

Accordingly, it is the basic object of the present invention to provide a guarded surgical scalpel which: 1) is practical; 2) easy and convenient to use; 3) relatively economical to manufacture; 4) meets all requirements of OSHA or other agencies; 5) can be used with standard, conventional surgical blades; 6) will facilitate the mounting (and subsequent de-mounting) of the blade on the scalpel; 7) will prevent the accidental or inadvertent de-mounting of the used blade during a surgical procedure in the O.R.; 8) will avoid any forceful ejection of a used blade; and 9) may be easily and conveniently taken apart for subsequent cleaning and sterilization.

The present invention finds particular utility in the environment of a guarded surgical scalpel of the type having a blade carrier slidably received within a housing. The housing functions as a handle for the scalpel and as a guard for a blade, the blade being adapted to be mounted on a cleat formed on the blade carrier. The blade, which is conventional, has a closed mounting slot formed therein, and this closed mounting slot includes a narrow forward portion and a wide rearward portion communicating therewith.

Within this environment, and in accordance with the teachings of the present invention, a hook member is carried by the blade carrier for conjoint movement therewith. This hook member, additionally, has a limited sliding movement relative to the blade carrier and has a laterally-projecting hook. The blade is placed on the scalpel such that the hook on the hook member and the cleat on the blade carrier are both received in the wide rearward portion of the closed mounting slot in the blade, and such that the cleat on the blade carrier is disposed forwardly of the hook on the hook member. Means are provided for moving the hook member rearwardly with respect to the blade carrier, such that the hook on the hook member engages the closed mounting slot in the blade and moves the blade conjointly therewith, and such that the cleat on the blade carrier is received in the narrow forward portion of the closed mounting slot in the blade, thereby securely and removably mounting the blade on the scalpel.

In the preferred embodiment, the blade has a rearward edge, and the hook member has a complementary shoulder formed thereon rearwardly of the hook. Means are provided for moving the hook member forwardly with respect to the blade carrier, such that the shoulder on the hook member engages the complementary rearward edge of the blade and pushes the blade off of the cleat on the blade carrier, thereby de-mounting the blade off the scalpel.

Preferably, the means for alternately moving the hook rearwardly to mount the blade and forwardly to de-mount the blade comprises a slider button having a laterally-extending stud (or equivalent portion) slidably received in a longitudinal slot formed in one side (preferably, the left side) of the housing. The slider button, in turn, is connected to the hook member.

Preferably, the means to connect the slider button to the hook member comprises a slider lock having a forward portion, a rearward boss portion, and an intermediate strap portion. The laterally-extending stud on the slider button is connected to the rearward boss on the slider lock; and fastening means connect the slider lock to the hook member, such that the blade carrier is sandwiched between the hook member and the slider lock.

The longitudinal slot in the side of the housing includes a forwardly-disposed stepped portion, such that the laterally-extending portion of the slider button is moved forwardly and upwardly and forwardly again, in sequence and in a substantially “z” configuration to literally push the blade off the scalpel in the blade-demounting process. There is thus no necessity for lifting the back of the blade laterally away from the blade carrier.

The laterally-projecting stud on the slider button is in the stepped portion of the longitudinal slot on the side of the housing and is moved rearwardly thereof to mount the blade on the scalpel.

In the preferred embodiment, the intermediate strap portion of the slider blade may flex within spaced-apart substantially horizontal planes to accommodate the “z” movement of the slider button.

The cleat on the blade carrier, being conventional, has respective portions, each of which is provided with a slit formed therein. These slits are substantially aligned in the same plane, such that when the cleat is in the narrow forward portion of the closed mounting slot in the blade, the blade has respective adjacent portions received in the slits in the cleat.

Another feature of the present invention is this: The blade carrier, hook member, slider lock and slider button form an integral subassembly. The other (right) side of the housing has an elongated opening formed therein, and a removable cover is provided for this elongated opening. The cover may be removed, and the subassembly may be angled slightly and withdrawn from the housing for subsequent cleaning and/or sterilization of the housing and subassembly, respectively.

Viewed in another aspect, the present invention provides a unique method of mounting a blade on to a blade carrier of a guarded surgical scalpel, wherein the blade carrier is slidably received within a housing, the housing forming a handle for the scalpel and a guard for the blade; and wherein the blade has a mounting slot formed therein, the mounting slot having a forward narrow portion and a rearward wider portion communicating therewith.

This improved method includes the steps of retracting the handle relative to the blade carrier, such that a forward portion of the blade carrier projects forwardly of the handle. A member is provided which is carried by the blade carrier and slidably movable relative thereto. This member has a first element thereon, and the blade carrier has a second element thereon. The second element has respective portions, each of them having a slit formed therein, the respective slits being substantially aligned. The blade is placed on the scalpel, such that the first element on the member is disposed in the rearward wider portion of the mounting slot in the blade, and such that the second element on the blade carrier is disposed in the wider portion of the mounting slot in the blade and is disposed forwardly of the first element on the member. The member is moved rearwardly relative to the blade carrier, such that the blade is thus moved rearwardly relative to the blade carrier, and such that the second element on the blade carrier is moved forwardly relative to the blade—the blade being moved rearwardly with respect to the blade carrier—to enable the second element to be moved out of the rearward wider portion of the mounting slot in the blade and to be received in the narrow portion of the mounting slot in the blade. In this position, respective slits in the second element on the blade carrier receive the respective portions of the blade body adjacent to the forward narrow portion of the mounting slot in the blade.

Preferably, the member comprises a hook member, the first element comprises a hook on the hook member, and the second element comprises a cleat on the blade carrier.

Viewed in yet another aspect, the present invention provides an improved method of, first, mounting a (new) blade on a surgical scalpel; second, using the scalpel safely during the surgical procedure; and, third, subsequently de-mounting the (used) blade off the scalpel. This improved method comprises the steps of providing a blade carrier telescopically received within a housing, and further providing a member carried by the blade carrier and independently movable relative thereto. The blade carrier is extended forwardly of the housing. A blade is provided and placed conjointly on the member and on the blade carrier, respectively. The member is moved rearwardly relative to the blade carrier to mount the blade securely on to the blade carrier. During a surgical procedure, the blade carrier is advanced relative to the housing for exposing the blade for use during the surgical procedure; and the blade carrier is retracted within the housing, thereby covering the blade for transfer of the scalpel from a surgeon to an assistant during the surgical procedure and vice-versa. The blade carrier is again advanced relative to the housing upon completion of (at least) a portion of the surgical procedure, and the member is moved forwardly relative to the blade carrier to de-mount the blade from the scalpel.

As will be appreciated by those skilled in the art, one basic concept of the present invention is the combination of a handle provided with a blade carrier having a forward portion provided with a cleat formed thereon, and a member carried by the blade carrier and slidably movable relative thereto, such that the member has a forward position and further has a rearward position relative to the blade carrier; and manually manipulatable means are carried by the handle for moving the member rearwardly into its rearward position for mounting a surgical blade on to the cleat on the forward portion of the blade carrier, thereby mounting the surgical blade on the scalpel. The manually-manipulatable means then moves the member forwardly into its forward position for subsequently demounting the surgical blade off the scalpel.

These and other objects of the present invention will be readily appreciated from the following specification taken in conjunction with the enclosed drawings.

### BRIEF DESCRIPTION OF THE DRAWINGS

FIG. 1 is a left side perspective of the guarded surgical scalpel of the present invention.

FIG. 2 is a right side perspective thereof.

FIG. 3 is a left side elevational view thereof.

FIG. 4 is a right side elevational view thereof.

FIG. 5 is a top plan view thereof.

FIG. 6 is an exploded perspective thereof.

FIGS. 7, 8 and 9 are respective orthogonal projections of the housing of the guarded surgical scalpel.

FIGS. 10, 11 and 12 are respective orthogonal projections of the blade carrier.

FIGS. 13, 14 and 15 are respective orthogonal projections of the hook member.

FIGS. 16, 17 and 18 are respective orthogonal projections of the slider lock.

FIGS. 19, 20 and 21 are respective orthogonal projections of the slider button.

FIG. 22 is a cross-section, taken along the lines 22-22 of FIG. 5, showing the blade mounted on the cleat on the blade carrier, and drawn to an enlarged scale.

FIG. 23 is an end view showing the guarded surgical scalpel viewed from the end of the blade looking into the scalpel.

FIG. 24 is a cross-section, taken along the lines 24-24 of FIG. 5, showing the slider lock connected to the hook member, and drawn to an enlarged scale.

FIG. 25 is a cross-section, taken along the lines 25-25 of FIG. 5, showing the detent button sliding in a slot formed in the top wall of the housing, and drawn to an enlarged scale.

FIGS. 26-29 are respective schematic sequence views, showing the mounting of the (new) blade on the scalpel.

FIG. 30 is a side elevation of the scalpel with the blade fully mounted, and with the blade carrier retracted in its guarded position.

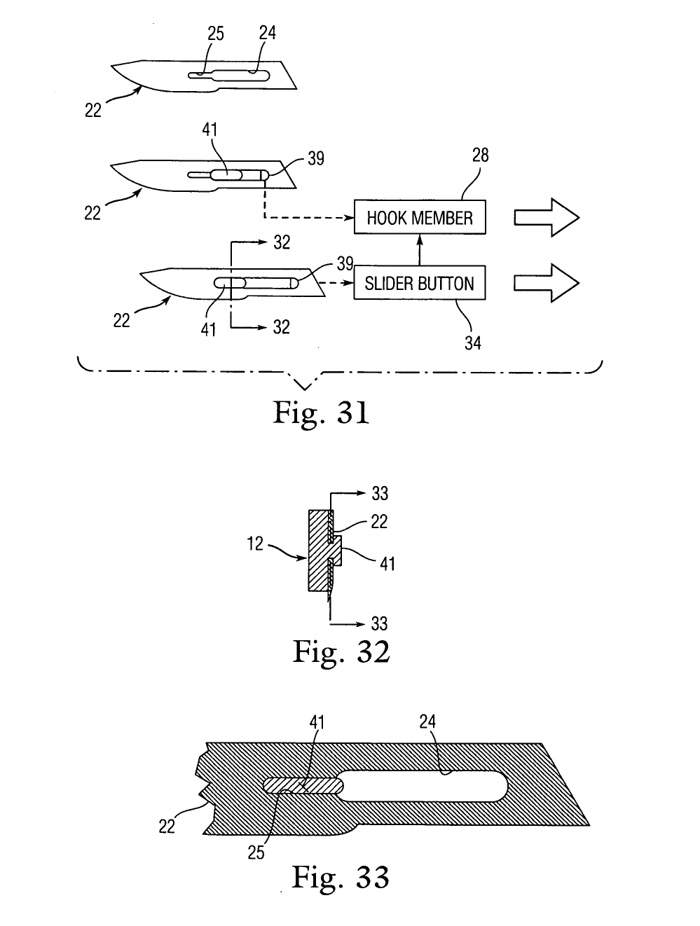

FIGS. 31-34 are respective schematic sequence views, showing the de-mounting of the (used) blade on the scalpel; FIG. 33 is a section view, taken along the lines 33-33 of FIG. 32, and drawn to an enlarged scale.

FIG. 35 is a flow chart, showing the method steps in mounting the blade on the scalpel.

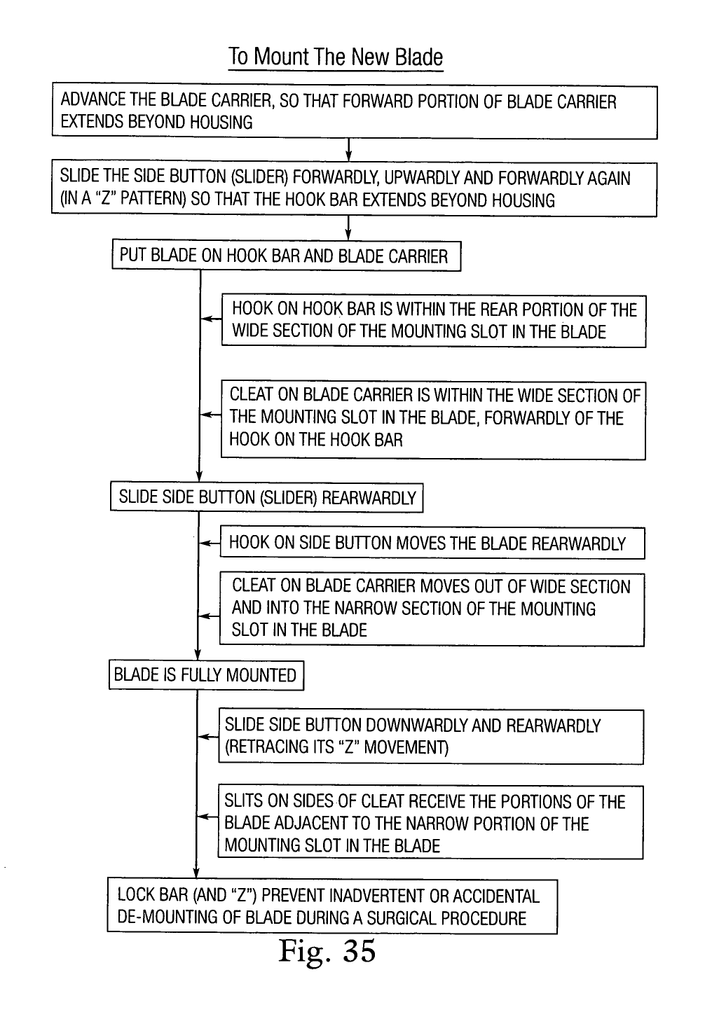

FIG. 36 is a flow chart, showing the method steps in de-mounting the blade on the scalpel.

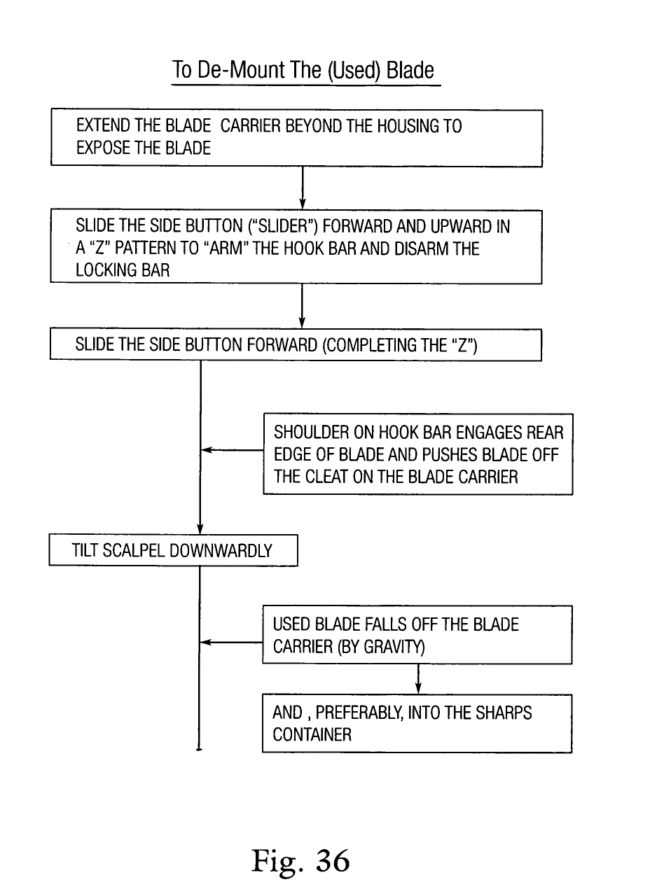

FIG. 37 is a right side perspective, corresponding substantially to FIG. 4, but showing the removable cover exploded away from the scalpel, and further showing the sub-assembly of the blade carrier, hook member, slider lock and slide button being angled slightly and removed from the housing for subsequent cleaning and sterilization of the sub-assembly and the housing, respectively.

FIG. 38 is a further exploded view, corresponding substantially to FIG. 6, but showing an alternate embodiment.

### GENERAL DESCRIPTION OF THE PREFERRED EMBODIMENTS

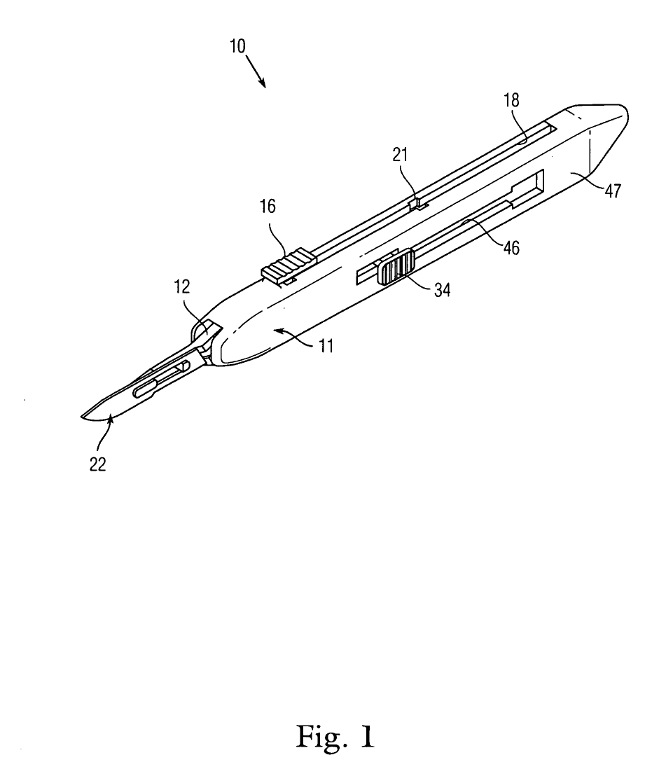

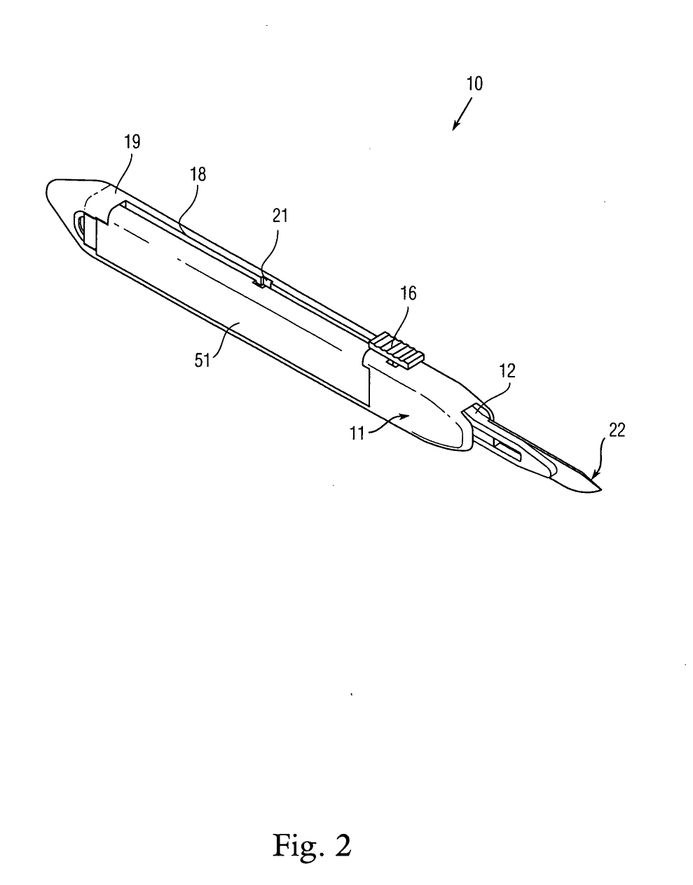

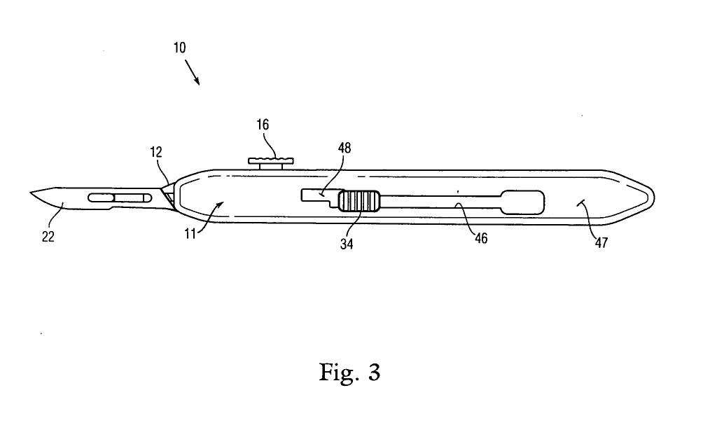

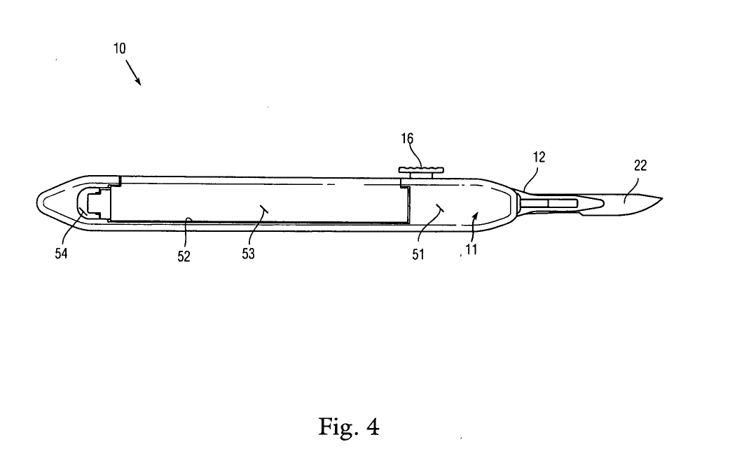

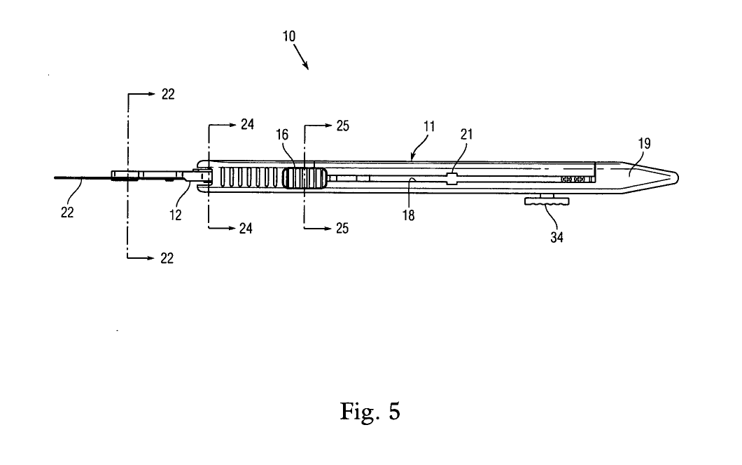

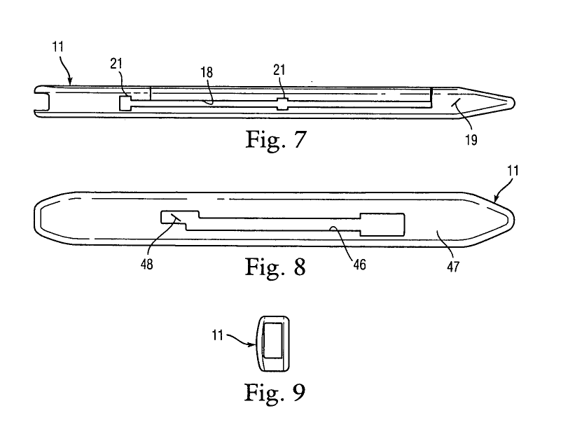

With reference to the drawings, and particularly to FIGS. 1-6, the guarded surgical scalpel 10 of the present invention has a housing 11 forming a handle for the scalpel 10, and a blade carrier 12 is slidably received, telescopically, within the housing 11. The blade carrier 12 has two alternate positions: advanced and retracted, respectively, relative to the housing 11. These alternate positions of the blade carrier 12 are controlled by a two-position resiliently-biased detent means 13 between the housing 11 and the blade carrier 12. This detent means 13 includes a cantilevered spring 14 (or its equivalent) secured to the blade carrier 12 by screws 15 (or other suitable means). A detent button 16 is secured to the cantilevered spring 14 by a screw 17 (or its equivalent). The detent button 16 slides within a slot 18 formed in the top wall 19 of the housing 11, and the detent button 16 has a laterally-projecting stud 20 adapted to be received, alternately, in one of the two detent pockets 21 formed in the housing 11 (see FIG. 7).

A conventional surgical blade 22 is carried by the blade carrier 12. In the advanced position of the blade carrier 12 (relative to the housing 11) the blade 22 is exposed; and in the retracted position of the blade carrier 12 (again, relative to the housing 11) the blade 22 is covered by the housing 11, the housing 11 thus forming a guard for the blade 22. [Basically, this is the general configuration of a guarded surgical scalpel of the type pioneered and advocated by Messrs. Lehmbeck and Abidin for quite some time.]

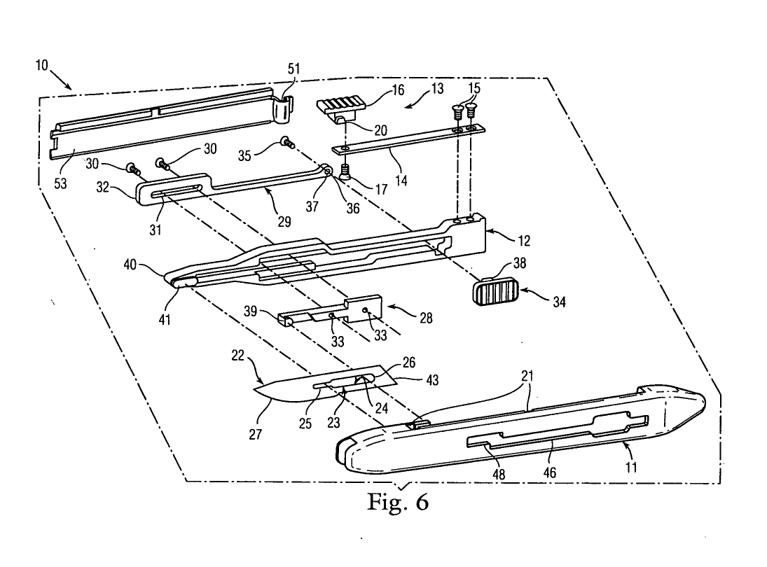

The surgical blade 22 has been standardized, basically, throughout the world and for many, many years. The blade 22 has a closed mounting slot 23 formed therein. This closed mounting slot 23 has a wide rearward portion 24 communicating with a narrow forward portion 25 (see FIG. 6). The wide rearward portion 24 terminates rearwardly in a wall 26, and the blade 22 has a cutting edge 27. Again, this configuration of the blade 22 is universal.

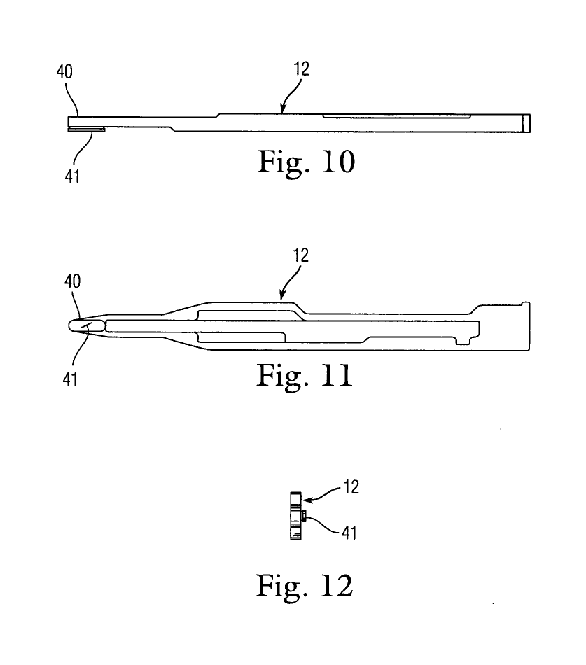

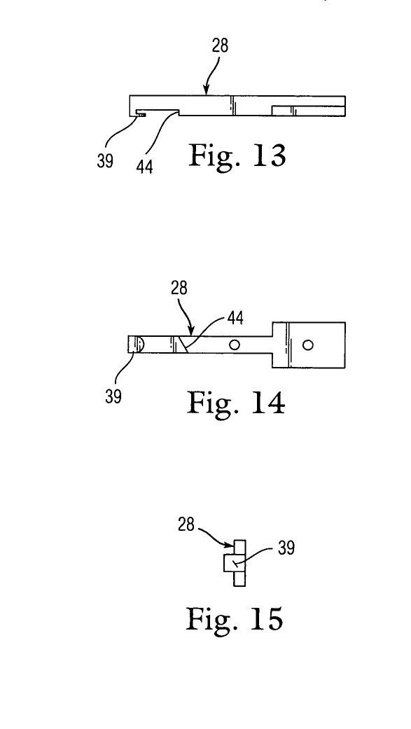

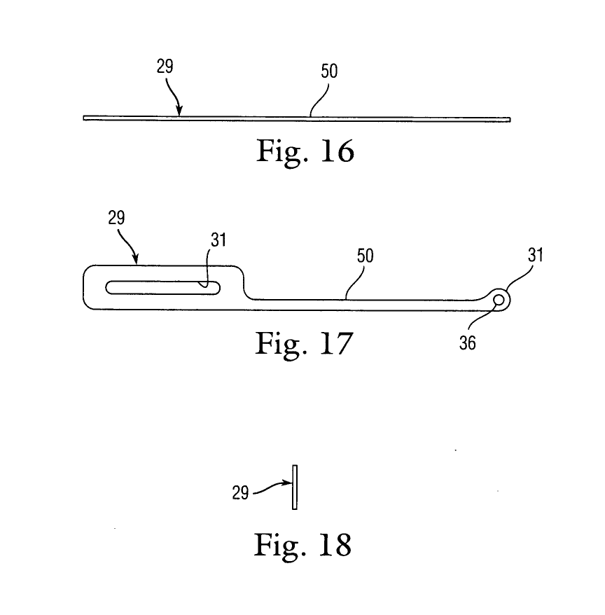

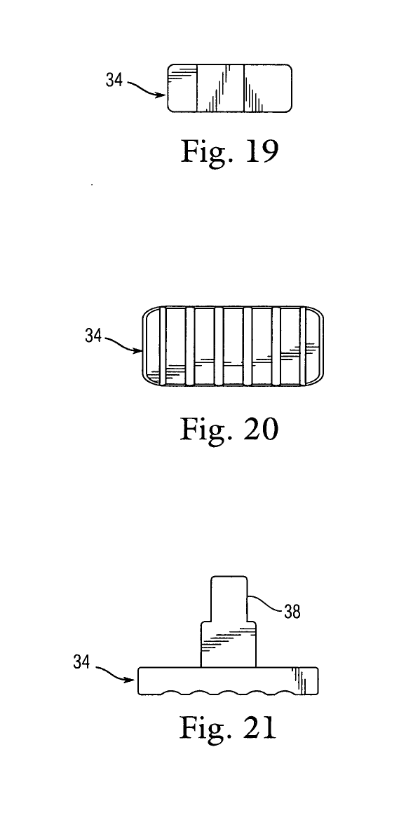

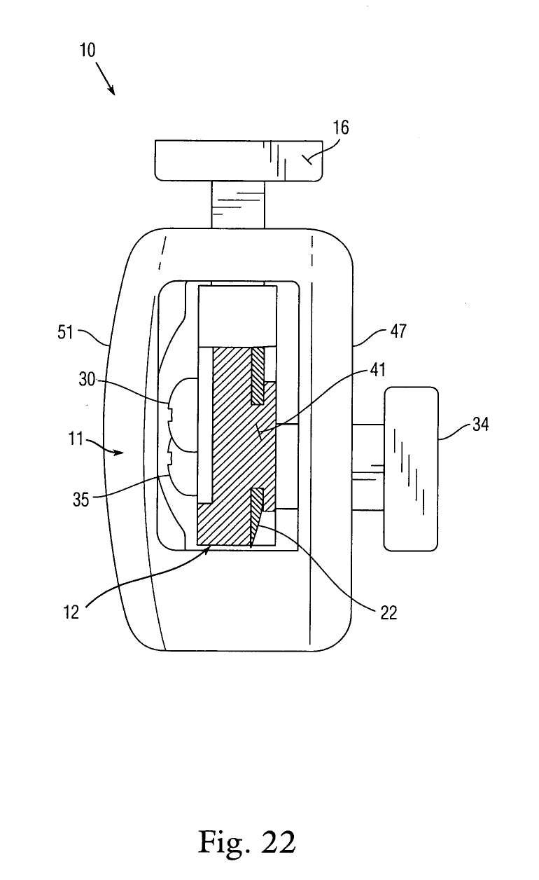

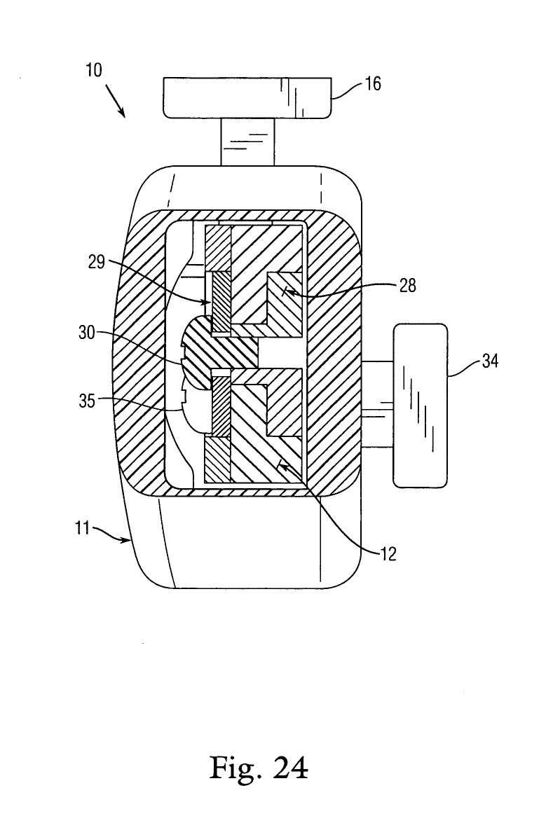

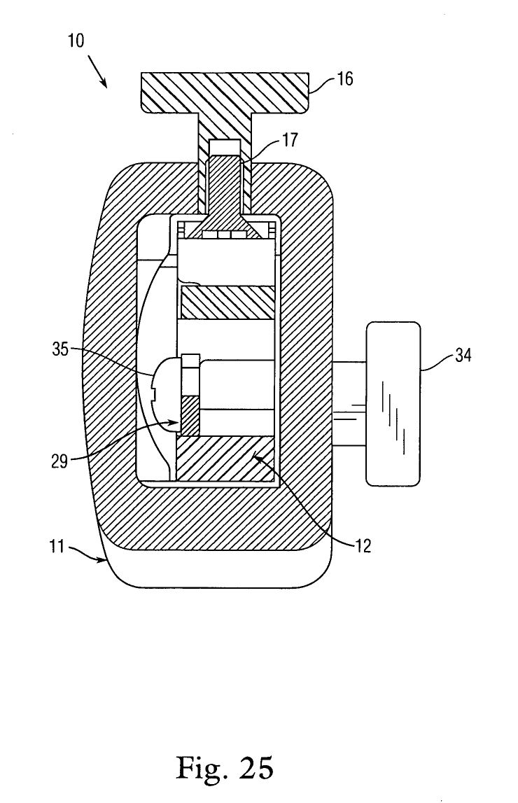

Now, in accordance with the teachings of the present invention, and with further reference to FIGS. 7-25, a hook member 28 is disposed on one side of the blade carrier 12 (the left side of the blade carrier 12 looking forwardly of the scalpel 10) and a slider lock 29 is disposed on the other side of the blade carrier 12 (the right side of the blade carrier 12, again looking forwardly of the scalpel 10). Thus, the blade carrier 12 is “sandwiched” between the hook member 28 and the slider lock 29 (see FIGS. 16-18) such that the slider lock 29 forms a clamp for the hook member 28. The slider lock 29 is connected to the hook member 28 by screws 30 (or their equivalent) which pass through a slotted opening 31 formed in an enlarged portion 32 of the slider lock 29 and are received in respective tapped holes 33 in the hook member 28. This elongated opening 31 reduces the overall weight of the scalpel 10.

The hook member 28, which is carried by the blade carrier 12 for conjoint sliding movement telescopically within the housing 11, nevertheless has a limited sliding movement relative to the blade carrier 12.

Preferably, but not necessarily, the blade carrier 12, hook member 28 and slider lock 29 are formed of surgical-grade stainless steel, while the housing 11 is molded of a suitable impact resistant plastic material. In an intended commercial embodiment of the present invention, the housing is molded in an attractive silver color, but any other color would suffice.

A slider button 34 is carried by on the housing 12. A screw 35 (or its equivalent) passes through a hole 36 in an enlarged rear boss 37 (on the slider lock 29) and the screw 35 is received in a tapped boss 38 on the slider button 34. Accordingly, the slider button 34, slider lock 29 and hook member 28 form a unitary sub-assembly carried by the blade carrier 12 and having a conjoint limited sliding movement thereon.

The hook member 28 has a laterally-projecting protuberance comprising a hook 39 formed forwardly thereon. The blade carrier 12, in turn, has a forward portion 40 provided with a laterally-projecting protuberance comprising a cleat 41 formed thereon.

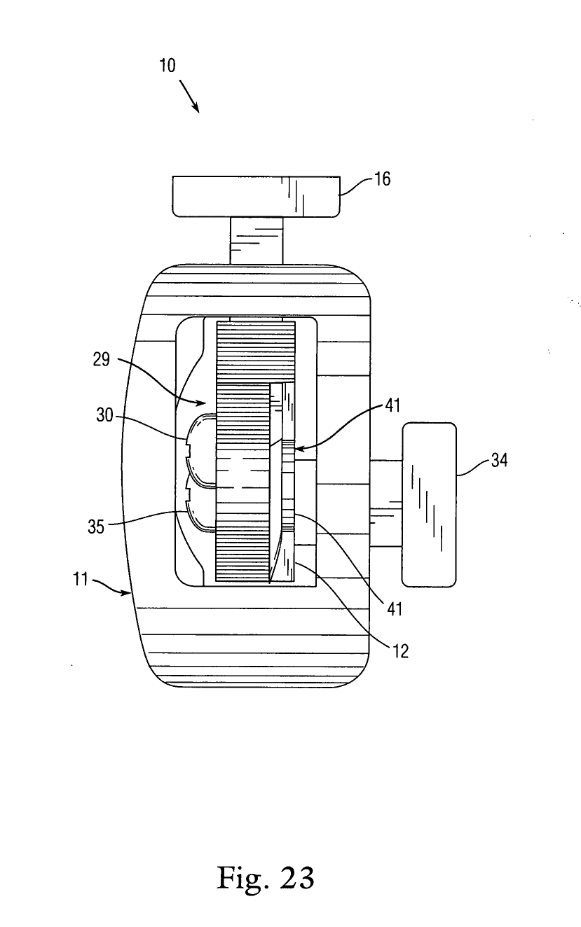

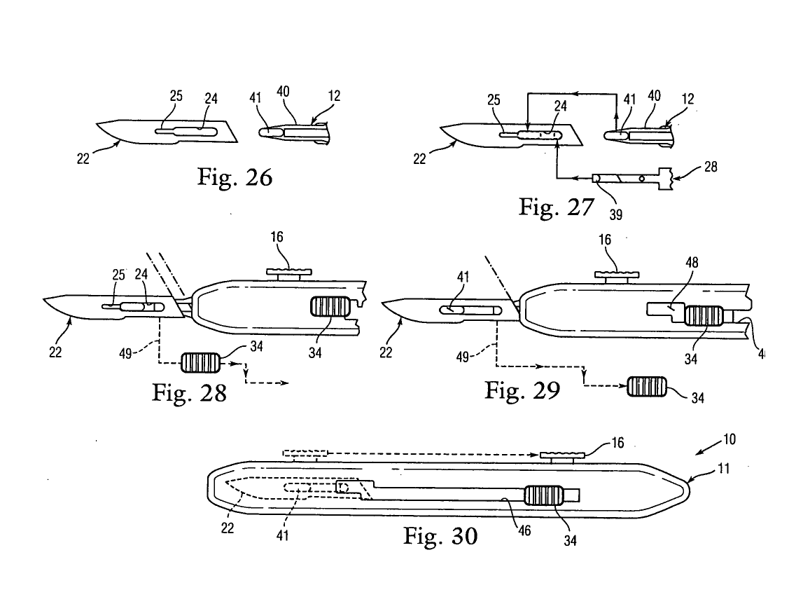

Accordingly, and with further reference to FIGS. 26-29, the operation of the blade mounting means of the present invention is as follows: The two-position detent means including the detent button 16 is manually engaged to extend the forward portion 40 of the blade carrier 12 beyond the housing 11. The blade 22 is placed on the scalpel 10, such that the hook 39 on the hook member 28 is received towards the back of the wide rearward portion 24 of the closed mounting slot 23 in the blade 22, and such that the cleat 41 on the forward portion 40 of the blade carrier 12 is also received in the wide rearward portion 24 of the closed mounting slot 23 in the blade and forwardly of the hook 39 (see FIG. 23). The slider button 34 is moved into its forwardly and upwardly (“armed”) position, as hereafter described, and the slider button 34 is then slid rearwardly (FIG. 28) to thus slide the hook member 28 rearwardly with respect to the blade carrier 12. In this regard, the hook 39 on the hook member 28 engages the wall 26 of the blade 22. As a result, the blade 22 is pulled rearwardly to enable the cleat 41 on the forward portion 40 of the blade carrier 12 to slip out of the wide rearward portion of the closed mounting slot 23 of the blade 22 and into the narrow forward portion thereof (FIG. 29) to securely (but removably) mount the blade 22 on the scalpel 10. As more clearly shown in FIG. 32, each of the sides of the cleat 41 has a slit 42 which receives the body of the blade 22.

In its mounted position on the scalpel 10, the rearward edge 43 of the blade 22 is disposed adjacent to a complementary shoulder 44 formed on the hook member 28. Preferably, and as shown more clearly in FIG. 6, the complementary rearwardly edge 43 of the blade 22 and the shoulder 44 on the hook member 28 are angled with respect to the longitudinal axis of the scalpel 10.

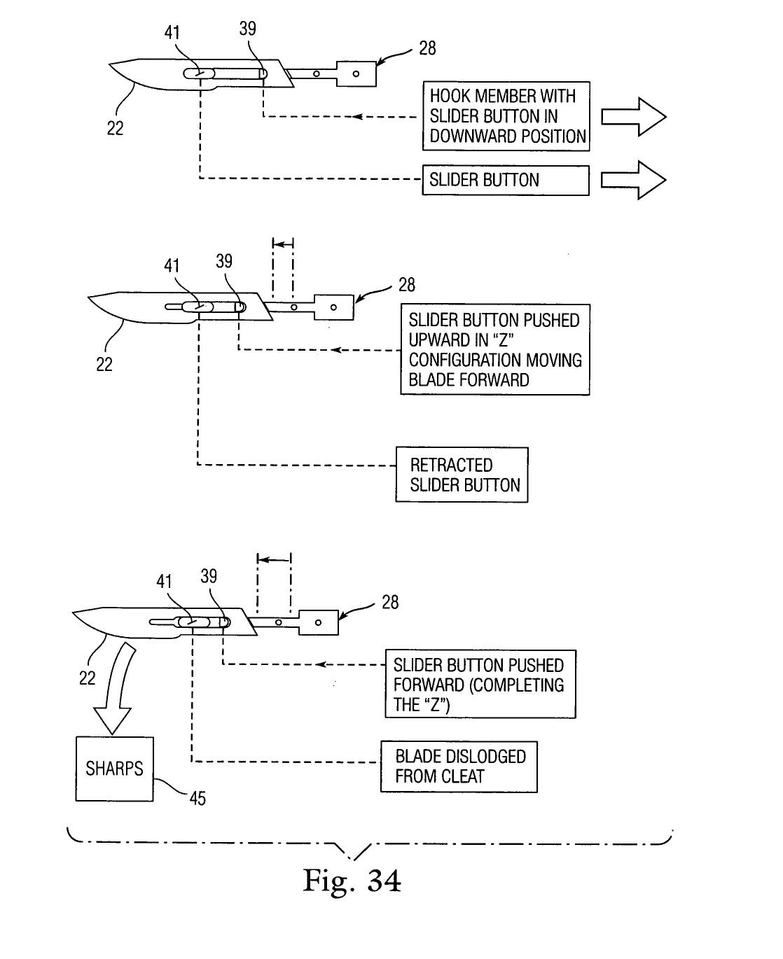

With respect to FIGS. 31-34, to de-mount the blade 22 from the scalpel 10, the slider button 34 is again “armed” in its forwardly and upwardly position (as discussed hereinafter) and the slider button 34 is pushed forwardly, thereby pushing the hook member 28 forwardly, such that the shoulder 44 on the hook member 28 engages the complementary rearward edge 43 on the blade 22 and pushes the blade off of the cleat 41 on the blade carrier 12. Thereafter, the blade 22 may merely fall off the scalpel 10 (by gravity) and, if preferred, into a sharps container 45 (FIG. 34).

The slider button 34 slides within an elongated slot 46 in the left side wall 47 in the housing 11. The elongated slot 46 terminates forwardly in a forward portion 48 having substantially a “z” configuration. As a result, the slider button 34 is moved forwardly and then upwardly (into an “armed” position) and then forwardly again to complete the substantially “z” movement 49 (FIGS. 28 and 29) of the slider button 34. As a result, the hook member 28 pushes the blade 22 off the scalpel 10. This “z” movement of the slider button 34 thus prevents an accidental or inadvertent activation of the slider button 34 which, otherwise, may push the blade 22 off the scalpel 10 while the scalpel 10 is being used during a surgical procedure.

To accommodate this “z” movement of the slider button 34, the slider lock 29 has an intermediate strap portion 50 which may flex slightly between substantially parallel spaced-apart vertical planes (not shown).

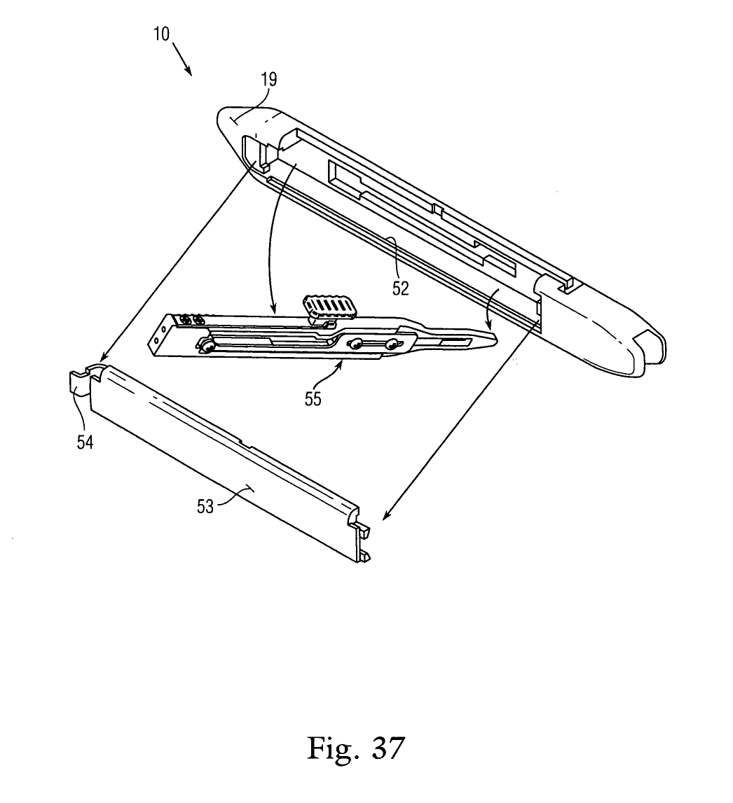

The housing 11 has a right side wall 51 (FIG. 37) which has an elongated opening 52 formed therein, and this elongated opening 52 is provided with a removable cover 53 having a spring latch portion 54. By actuating the latch 54, the cover 53 may be removed from the housing 11. Thereafter, the entire sub-assembly 55—of the blade carrier 12, hook member 28, slider lock 29 and slider button 34—may be angled and removed out of the opening 52 in the housing 11 for subsequent cleaning and/or sterilization of the sub-assembly 55 and the housing 11, respectively, quickly and conveniently. This is yet another feature and advantage of the present invention.

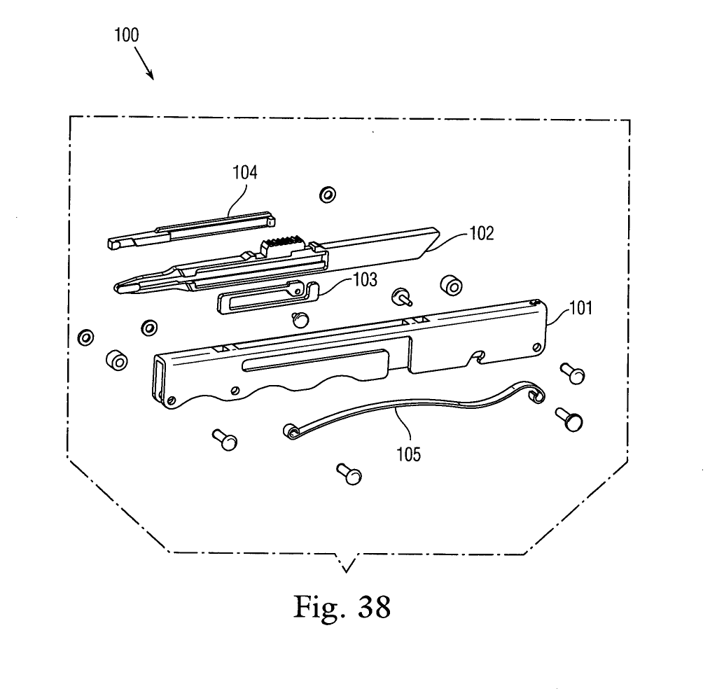

With reference to FIG. 38, an alternate embodiment of the present invention has a scalpel 100 with a housing (guard) 101 telescopically receiving a blade carrier 102. The blade carrier 102 is “sandwiched” between the (revised) hook member 103 and the (revised) slider lock 104. The operation of the scalpel 100 is similar to the operation of the preferred embodiment comprising the scalpel 10 (except for the “z” operation of the slider button) and the removal of the blade carrier 102, hook member 103 and slider lock 104 as an integral subassembly following a surgical procedure for cleaning and/or sterilization. Rather, disassembly is facilitated by a spring latch 105, such that the subassembly comes out of an opening in the bottom of the housing 101.

Obviously, many modifications may be made without departing from the scope and intent of the present invention. For example, in lieu of the hook 39 on the hook member or bar 28, a post could be employed to move the blade 22 rearwardly on the blade carrier 12. Accordingly, within the appended claims, the invention may be practiced other than specifically disclosed herein.

What is claimed is:

1. In a guarded surgical scalpel having a blade carrier slidably received within a housing, the housing functioning as a handle for the scalpel, and further functioning as a guard for a blade adapted to be mounted on a cleat formed on the blade carrier, the blade having a closed mounting slot formed therein, including a narrow forward portion and a wide rearward portion communicating therewith, the improvement comprising a hook member carried by the blade carrier for conjoint movement therewith and having a limited sliding movement relative to the blade carrier, the hook member having a laterally projecting hook, the blade being placed on the scalpel such that the hook on the hook member and the cleat on the blade carrier are both received in the wide rearward portion of the closed mounting slot in the blade, and such that the cleat is disposed forwardly of the hook, and means for moving the hook member rearwardly with respect to the blade carrier, such that the hook on the hook member engages the closed mounting slot in the blade and moves the blade conjointly therewith, and such that the cleat on the blade carrier is received in the narrow forward portion of the closed mounting slot in the blade, thereby removably mounting the blade on the scalpel.

2. The improvement of claim 1, wherein the blade has a rearward edge, and wherein the hook member has a shoulder formed thereon rearwardly of the hook, and means for moving the hook member forwardly with respect to the blade carrier, such that the shoulder on the hook member engages the rearward edge of the blade and pushes the blade off of the cleat on the blade carrier, thereby de-mounting the blade off the scalpel.

3. The improvement of claim 1, wherein the means for alternately moving the hook rearwardly to mount the blade and fowardly to de-mount the blade comprises a slider button having a laterally-extending portion slidably received in a longitudinal slot formed in one side of the housing, and means for connecting the slider button to the hook member.

4. The improvement of claim 3, wherein the means to connect the slider button to the hook member comprises a slider lock having a forward portion, a rearward boss portion, and an intermediate strap portion, the laterally-extending portion of the slider button being connected to the rearward boss on the slider lock, and fastening means connecting the slider lock to the hook member, the blade carrier being sandwiched between the hook member and the slider lock.

5. The improvement of claim 4, wherein the longitudinal slot in the side of the housing includes a forwardly-disposed stepped portion, such that the laterally-extending portion of the slider button is moved forwardly and upwardly and forwardly again, in sequence and in a substantially “z” configuration to push the blade off the scalpel.

6. The improvement of claim 5, wherein the laterally-projecting portion of the slider button is in the stepped portion of the longitudinal slot on the side of the housing and is moved rearwardly thereof to mount the blade on the scalpel.

7. The improvement of claim 5, wherein the intermediate strap portion of the slider blade may flex within spaced-apart substantially horizontal planes to accommodate the “z” movement of the slider button.

8. The improvement of claim 1, wherein the cleat on the blade carrier has respective portions, each of which is provided with a slit formed therein, the slits being substantially aligned in the same plane, such that when the cleat is in the narrow forward portion of the closed mounting slot in the blade, the blade has respective adjacent portions received in the slits in the cleat.

9. The improvement of claim 1, wherein the blade carrier, hook member, slider lock and slider button form an integral subassembly, and wherein a side of the housing has an elongated opening formed therein, and a removable cover for the elongated opening, such that the cover may be removed, and such that the subassembly may be withdrawn from the housing for cleaning and/or sterilization of the housing and subassembly, respectively.

10. A guarded surgical scalpel, comprising a housing forming a handle for the scalpel, a blade carrier slidably received within the housing and adapted to have a blade mounted thereon, two-position manually-manipulatable detent means for alternately advancing and retracting the blade carrier relative to the housing; such that in the advanced position of the blade carrier the blade is exposed, and such that in the retracted position of the blade carrier the housing forms a guard for the blade, a hook member carried by the blade carrier and disposed on one side of the blade carrier, and having a limited sliding movement relative to the blade carrier, a slider lock on the other side of the blade carrier, the blade carrier being sandwiched between the hook member and the slider lock, a slide button on one side of the housing and connected to a rearward portion of the slider lock, the slider lock having an intermediate strap portion between the forward and rearward portions thereof, the strap portion being capable of limited flexing movement, the slide button sliding within a slot formed in a side wall of the housing, the slot having a forwardly disposed portion forming substantially a “z” configuration, such that the slide button is pushed forwardly, upwardly and forwardly again, in sequence, thereby arming the slide button, the blade having a mounting slot having a narrow forward portion communicating with a rearward wide portion, a hook on the hook member received in the wide portion of the mounting slot in the blade, and a cleat on the blade carrier received initially in the wide portion of the mounting slot in the blade and forwardly of the hook on the hook member, such that the slide button may be moved rearwardly to move the hook member and hence the blade rearwardly, and such that the cleat moves out of the wide portion of the mounting slot in the blade and into the narrow portion thereof, the cleat having respective top and bottom portions, each of which is provided with a slit therein, the slits engaging the blade adjacent to the narrow portion of the mounting slot in the blade, thereby mounting the blade on the scalpel.

11. A guarded surgical scalpel, comprising a blade carrier within a housing, the housing having a pair of sides and forming a handle for the scalpel and a guard for a blade mounted on the blade carrier, first means including a member carried by the blade carrier and having limited sliding movement relative thereto for alternately mounting and demounting the blade on the blade carrier, second means including a manually-manipulatable slider button on one side of the housing and carried by the blade carrier and movable relative thereto for actuating the member, the other side of the housing having a longitudinally-aligned opening formed therein, such that upon demounting the blade from the blade carrier following a surgical procedure, the blade carrier along with the first and second means carried thereon may be removed as a subassembly through the opening and out of the housing, thereby facilitating the cleaning and/or sterilization of the subassembly and the housing, respectively, and a removable cover for the longitudinally aligned slot in the housing.

12. The scalpel of claim 11 wherein the first element on the member comprises a hook, and wherein the second element on the blade carrier comprises a cleat.

13. The scalpel of claim 11, wherein the blade has a rearward edge, and wherein the member has a complementary shoulder formed therein.

14. The method of de-mounting the blade off the scalpel of claim 13 following a surgical procedure, comprising the steps of advancing the blade carrier relative to the housing such that the cleat on the blade carrier with the blade mounted thereon projects forwardly of the housing, and moving the slider button forwardly and relative to the blade carrier, such that the shoulder on the member engages the rearward edge of the blade and pushes the blade off of the cleat on the blade carrier.

15. The method of claim 14, further including the step of tilting the scalpel downwardly, such that the blade falls off the scalpel by reason of gravity.

16. A method of mounting a blade on to a blade carrier of a guarded surgical scalpel, wherein the blade carrier is slidably received within a housing, the housing forming a handle for the scalpel, and wherein the blade has a mounting slot formed therein, the mounting slot having a forward narrow portion and a rearward wider portion communicating therewith, comprising the steps of retracting the handle relative to the blade carrier, such that a forward portion of the blade carrier projects forwardly of the handle, providing a member carried by the blade carrier and slidably movable relative thereto, the member having a first element thereon, and the blade carrier having a second element thereon, the second element having respective portions, each of them having a slit formed therein, the respective slits being substantially aligned, placing the blade on the scalpel such that the first element on the member is disposed in the rearward wider portion of the mounting slot in the blade, and such that the second element on the blade carrier is disposed in the wider portion of the mounting slot in the blade and is disposed forwardly of the first element on the member, and moving the member rearwardly relative to the blade carrier, such that the blade is moved rearwardly relative to the blade carrier, and such that the second element on the blade carrier is moved forwardly relative to the blade to enable the second element to be moved out of the rearward wider portion of the mounting slot in the blade and to be received in the narrow portion of the mounting slot in the blade, the respective slits in the second element on the blade carrier receiving the respective portions of the blade adjacent to the forward narrow portion of the mounting slot in the blade.

17. The method of claim 16, wherein the member comprises a hook member, wherein the first element comprises a hook on the hook member, and wherein the second element comprises a cleat on the blade carrier.

18. A method of mounting a blade on a surgical scalpel, using the scalpel safely and subsequently de-mounting the blade off the scalpel, comprising the steps of providing a blade carrier telescopically received within a housing and further providing a member carried by the blade carrier and independently movable relative thereto, extending the blade carrier forwardly of the housing, providing a blade and placing the blade conjointly on the member and blade carrier, respectively, moving the member rearwardly relative to the blade carrier to move the blade securely on to the blade carrier, alternatively advancing the blade carrier relative to the housing for exposing the blade for use during a surgical procedure and retracting the blade carrier within the housing, thereby covering the blade for transfer of the scalpel from a surgeon to an assistant during the surgical procedure and vice-versa, advancing the blade carrier relative to the housing upon completion of a least a portion of the surgical procedure, and moving the member forwardly relative to the blade carrier to de-mount the blade from the scalpel.

19. In a surgical scalpel, the combination of a blade carrier having a forward portion provided with a cleat formed thereon, a hook member carried by the blade carrier and movable relative thereto, the hook member having a hook formed thereon, a blade adapted to be mounted on the blade carrier, the blade having a closed mounting slot formed therein, the closed mounting slot having a rearward wide portion terminating in a rearward edge and further having a forward narrow portion communicating with the rearward wide portion therein, the blade being initially received on the scalpel, such that the hook on the hook member is received within the rearward wide portion of the closed mounting slot in the blade, and such that the cleat on the blade carrier is also received within the rearward wide portion of the closed mounting slot in the blade and is disposed forwardly of the hook on the hook member, and means for moving the hook member rearwardly and independently of the blade carrier, such that the hook on the hook member engages the rearward edge of the closed mounting slot in the blade and pushes the blade conjointly with the hook member rearwardly relative to the blade carrier; and such that the cleat on the blade carrier is thereby received in the forward narrow portion of the closed mounting slot in the blade, thereby mounting the blade on the scalpel.

20. The combination of claim 19, further including a housing within which the blade carrier is slidably received, the housing forming a handle for the scalpel, and manually-manipulatable two-position detent means between the handle and the blade carrier; such that in one position the blade on the blade carrier is exposed forwardly of the housing; and such that in the other position, the blade is covered by the housing, the housing thereby forming a guard for the blade.

21. The combination of claim 19, wherein the means for moving the hook member includes a slider button received in a slot formed in a side wall of the housing, the slider button being connected to the hook member for conjoint sliding movement relative to the blade carrier.

22. The combination of claim 21, wherein the slot in the side wall of the housing has a forward portion requiring the slide button to move forwardly, upwardly and forwardly again in substantially a “z” pattern, to arm the slide button to activate the hook member, such that the slide button may thereafter be moved rearwardly in the top portion of the “z” configuration, thereby moving the hook member rearwardly.

23. The combination of the claim 19, wherein the hook member has a shoulder formed thereon, and wherein the blade has a rearward edge complementary to the shoulder on the hook member, the rearward edge of the blade being adapted to rest substantially against the shoulder on the hook member when the blade is mounted on the scalpel.

24. The combination of claim 23, wherein the shoulder on the hook member and the rearward edge of the blade are angled with respect to the longitudinal axis of the scalpel.

25. The combination of claim 22, wherein the slider button may be moved forwardly, such that the shoulder on the hook member engages the rearward edge of the blade and pushes the blade off the scalpel.

26. In a surgical scalpel, the combination of a handle provided with a blade carrier having a forward portion provided with a cleat formed thereon, a member carried by the blade carrier and slidably movable relative thereto, such that the member has a forward position and further has a rearward position relative to the blade carrier, and manually manipulatable means carried by the handle for moving the member rearwardly into its rearward position for mounting a surgical blade on to the cleat on the forward portion of the blade carrier and thereby mounting the surgical blade on the scalpel, said manually-manipulatable means moving the member forwardly into its forward position for subsequently demounting the surgical blade off the scalpel.
